(19) United States
(12) Patent Application Publication
(10) Pub. No.: US 2022/0047758 A1
(43) Pub. Date: Feb. 17, 2022

(54) Portable Air Sterilizer for Respirators Used in Infectious Environments

(71) Applicants: Cazemir-Benedict Ionescu, Vladiceasca (RO); Radu Radu, Bucharest (RO)

(72) Inventors: Cazemir-Benedict Ionescu, Vladiceasca (RO); Radu Radu, Bucharest (RO)

(21) Appl. No.: 17/390,011

(22) Filed: Jul. 30, 2021

(30) Foreign Application Priority Data
Aug. 17, 2020 (RO) A202000520

(51) Int. Cl. A61L 9/16

(52) U.S. Cl. CPC A61L 9/16

(57) ABSTRACT

A portable air sterilization device is disclosed that attaches to respirators, and is used for the personal protection of individuals working in highly infectious environments for extended periods of time. The air sterilization device is portable and deactivates 99.99%-100% of aerosolized viruses and bacteria present in the infected air. To sterilize the infected air, the device uses the method of thermal stress.

This invention refers to a portable air sterilizer that attaches to personal protection respirators used by individuals working in infectious environments for extended periods of time.

The protection of personnel who work in infectious environments for long periods of time is only partially resolved by use of masks and respirators that filter the air to retain bacteria and viruses. Filters trap particles mechanically based on the spacing between the material fibers, and retain about 45-55% of particles larger than 1 micron in size. However, the dimensions of the SARS-COV-2 virus, which causes COVID-19, range between 0.14 and 0.016 microns. Reducing the spacing between the filter fibers to retain finer particles decreases the air flow through the filter, which may cause difficulty breathing, or make breathing impossible.

Retaining particles smaller than 1 micron has been possible due to the development of filtering media using electret technology. These fibers carry electrostatic loads on their surfaces, which allow them to attract and bind fine particles, even when the particle size is less than the distance between fibers. (JP2006528549)

All high-performance particulate filter respirators that comply with government health standards (i.e., N95, NK95, and FFP2) use electret-based filtering and retain at least 95% of particles larger than 0.3 microns. Also, filter-based respirators are not tightly sealed to the face and allow for an additional infiltration of up to 8% of the inhaled air volume. Therefore, even when performance masks that comply with N95, NK95 and FFP2 standards are used, a small amount of viruses are still likely to pass unfiltered into the body. When the environment is highly contaminated, and when exposure time is extensive (over days and weeks) these small viral loads can accumulate and trigger the disease.

The last generation of masks have addressed the air flow reduction through the electret filters by the addition of an electric-powered air pump/turbine (see U.S. Pat. No. 7,469,699B2). However, these higher performing masks still allow 1% of unfiltered viruses to enter the body, which can accumulate over time, triggering disease.

The use of the sterilized air generator as a component of a respirator results in a 99.99% to 100% inactivation rate of any viruses or bacteria. This means that the concentration of active viruses after passing through the air device is 100 times lower than the concentration of viruses that pass unfiltered when using the highest performing electret fiber masks. The sterilized air generator presented herein does not use filtration to retain viruses. It inactivates viruses and bacteria by rendering them unable to reproduce or transmit disease by the use of thermal stress.

Thermal stress requires a very short treatment time (between 1/10^{th} to 1/100^{th} of a second) to inactivate viruses. Thus, this inactivation method provides a feasible solution for the construction of a portable respirator.

Virus inactivation by use of UV-C radiation requires treatment times in the tens of seconds and generates compounds that are irritant to the airways, notably Ozone (O3) and Nitrous Oxides (NOx). Inactivation by high tension electric discharges in the air such as Corona or Dielectric Barrier Discharge Plasma requires shorter treatment times, but also generates Ozone (O3) and Nitrous Oxides (NOx).

Grinshpun et al.'s (2010) article “Inactivation of Aerosolized Viruses in Continuous Air Flow with Axial Heating,” established the conditions necessary to reach thermal stress, that inactivates viruses. They analyzed the degree of inactivation of the MS2 bacteriophage virus by varying the air temperature and treatment time. They achieved inactivation rates of 99.99% with treatment times in the 0.3 to 0.5 seconds, at a temperature of 175 degrees Celsius. Above this critical temperature, the necessary time for inactivation drops exponentially. All viruses are built from the same types of protein which make up the virus genome. As virus inactivation is achieved by steric degradation of the proteins that codify viral activity, the results observed in the MS2 virus study can be extrapolated to all other viruses.

The design of the sterilized air generator described in this patent fulfils the necessary conditions to ensure thermal stress and virus inactivation. The device ensures that at the minimum, an average volume of 8 liters of air per minute (which is normally required to breathe), is exposed to a temperature above 175 degrees Celsius in the treatment chamber for 0.3-0.5 seconds.

The goal of this invention is to design a portable air sterilizer that ensures a 99.99% inactivation rate of viruses and bacteria. The technical challenge that is overcome by this invention is attaining a high percentage of heat reclamation needed to treat the incoming air by thermal stress while maintaining a low thermal inertia for the device overall. The design of the air sterilizer provides a novel solution by placing the heat reactor inside the counter-current spiral-shaped heat exchanger. This eliminates the need for thermal insulation, which would have increased the device thermal inertia, and would have required a larger energy supply, negatively affecting device portability.

Reaching the thermal stress critical temperature of 175 degrees Celsius in a 3-5 minute timeframe demands reduced heat losses (a low dissipation of power on the electric resistance of the heat reactor) and reduced thermal inertia.

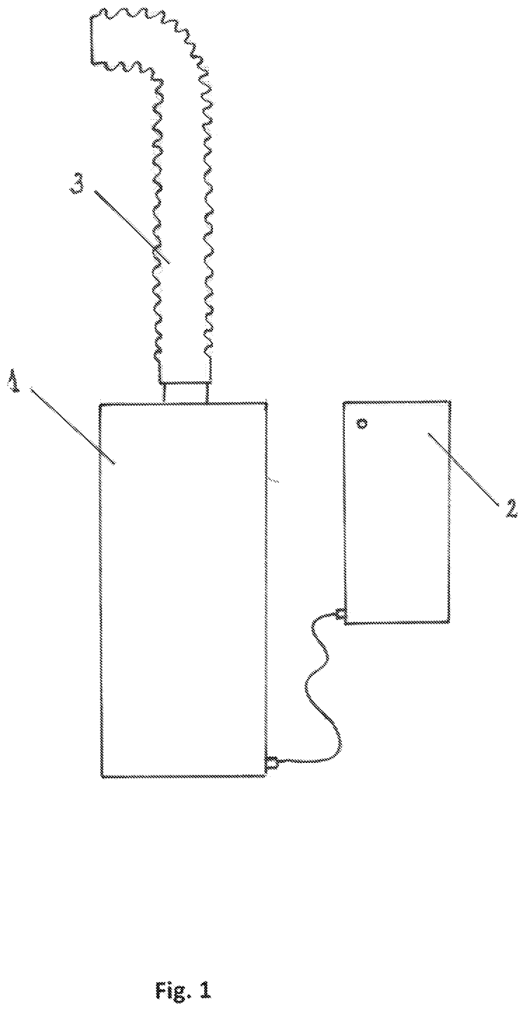

FIGS. 1-5 illustrating the design of the air sterilizer represent the following:

FIG. 1—Components schematics of the sterilized air generator in the first design version.

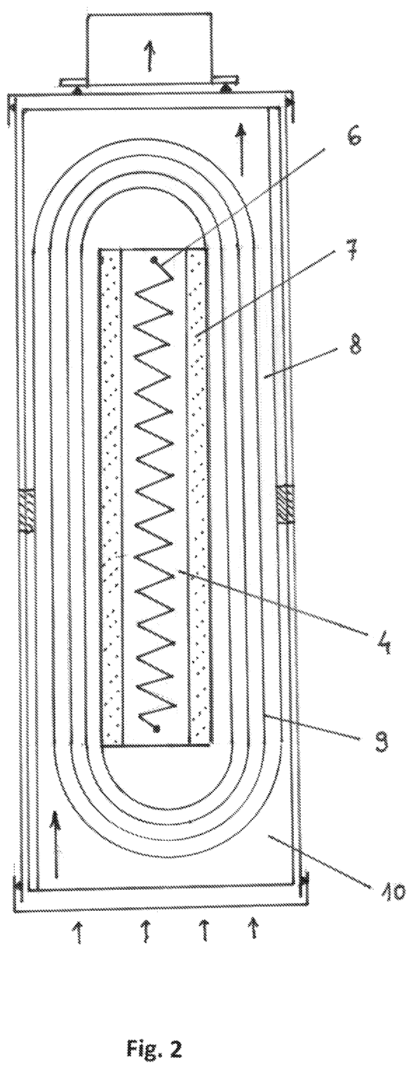

FIG. 2—Horizontal and longitudinal section through the thermal treatment chamber in the first design version.

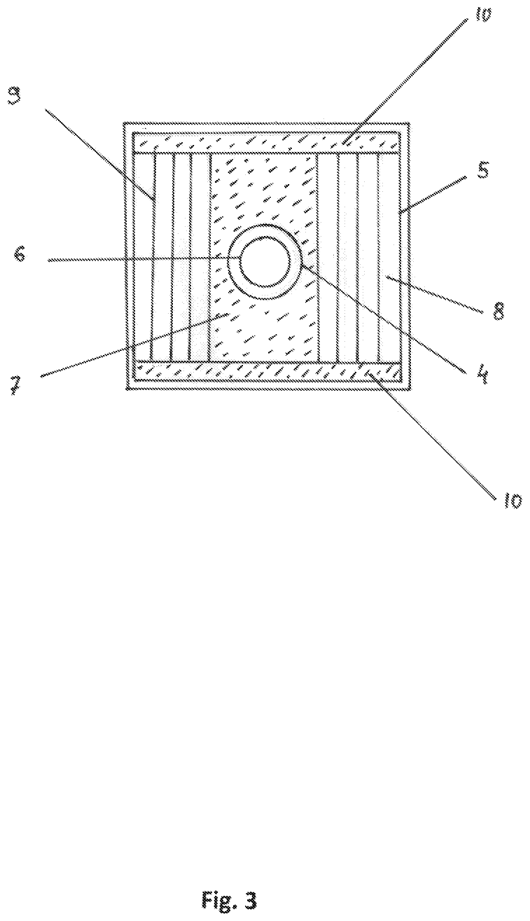

FIG. 3—Transverse section through the thermal treatment chamber in the first design version.

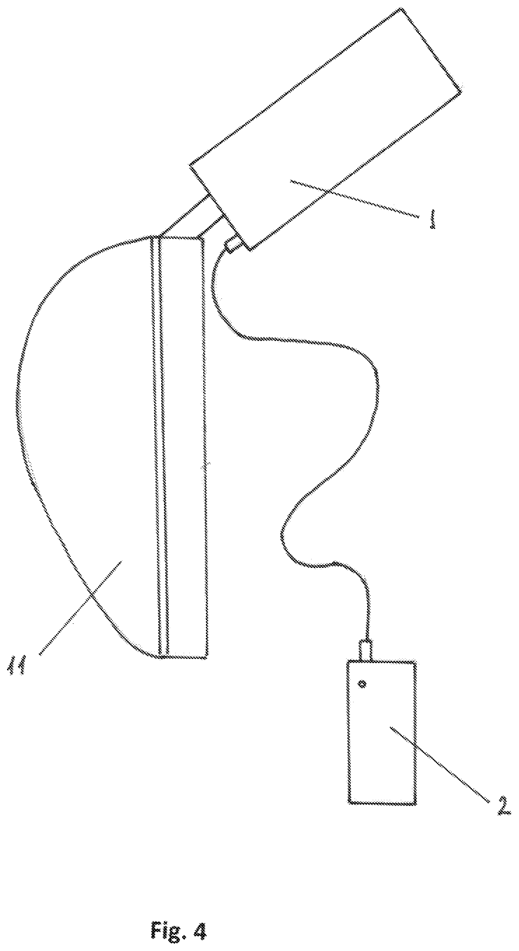

FIG. 4—Component schematics of the air sterilizer in the second design version.

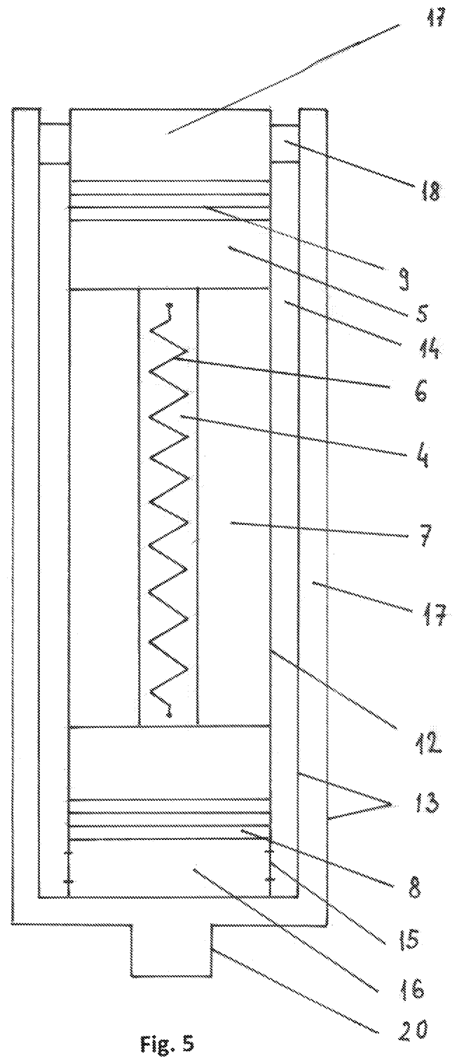

FIG. 5—Horizontal and longitudinal section through the thermal treatment chamber in the second design version.

FIG. 1 schematically shows the first version of the air sterilizer, composed of:

A. Thermal air treatment module (1)

B. Electrical power source (2)

C. Flexible hose (3) for feeding the sterilized air to the full-face mask (11)

FIG. 2 shows the thermal treatment module for the air (1) composed of the thermal treatment reactor (4) and counter-current spiral heat exchanger (5).

In the design version presented in FIG. 2, the spiral heat exchanger (5) surrounds the thermal treatment reactor (4). Thus, heat loss from the reactor (4) is used in pre-heating the air before treatment. This spatial arrangement eliminates the need for thermal insulation which would have increased the thermal inertia of the device.

A. The thermal air-treatment module (1) is composed of

a. A thermal treatment reactor (4) and
b. A spiral counter-current heat exchanger (5) which surrounds the thermal treatment reactor (4).

A.a. The thermal treatment reactor (4) was designed to reach a temperature equal to, or higher than 175 degrees Celsius in a short timeframe (3-5 minutes), while using minimal electrical power. The low power usage is possible by recovering the heat used in treatment and using it to pre-heat the infected air, and by reducing the thermal inertia of the thermal treatment reactor (4)/spiral counter-current heat exchanger (5) assembly. The heat losses in the thermal treatment reactor were reduced by placing the thermal treatment reactor (4) in the center of the spiral heat exchanger. This way, the heat lost by the reactor was used to pre-heat the air that is going to be treated. This spatial arrangement eliminated the need for additional thermal insulation, which would have increased the thermal inertia of the device.

The thermal treatment reactor is composed of a self-supporting Nikelin wire electrical resistance (6) mounted in a 17 mm diameter channel within a ceramic fiber bloc (7) with dimensions of 120 mm×25 mm×50 mm. The resistance coils (6) heat the air passing through the channel to a temperature above 175 degrees Celsius.

A.b. The spiral counter-current heat exchanger (5) with counter current air channels (8) arranged in a double-spiral cools the sterilized hot air while recovering the heat from the treated air to preheat the air going into the thermal treatment reactor. The two counter-current air channels (8) are formed between two sheets of aluminum (9) 50 mm wide by 110 mm long, rolled into a concentric spiral. The sheets are rolled while leaving a space of 3-6 mm between them. Two ceramic fiber lids (10) along with the aluminum sheets (9) form the two counter-current air channels (8). For reduced weight, small thermal inertia, and efficient heat exchange between the air channels (8), the aluminum sheets (9), only 0.07-0.1 mm thick, are corrugated transversally along their length. Corrugation of the aluminum sheets (9) ensures stability and rigidity of the final spiral shape and at the same time contributes to a turbulent airflow in the channels (8), increasing the thermal transfer between the air counter-currents.

B. Electrical Power Source (2)

The power source is composed or two 5000 mA Li-Ion rechargeable power cells, linked in series. Within the power source there is also a tension control and adjustment circuit as well as a LED charge indicator for the cells. Under proper usage within the 4.2V-3V range, the cells should provide 500-1000 charge-recharge cycles.

To extract the maximum energy from the cells without affecting their proper usage range (4.2V-3V) a tension lifting and regulating circuit was built into the source. The electrical power dissipated along the reactor (6) resistance, which assures that the treatment temperature stays above 175 degrees Celsius, is 15 W-12 W. The dissipated power depends on the tension (U) and the intensity (I) applied to the resistance: U(W)=U(V)×I(A). As the tension on the leads of the cells drops during operation, the power dissipated along the resistance also drops. If the power was initially sufficient to reach a temperature of 175 degrees Celsius in a short time (3-5 minutes) and to maintain it within the 200-210 degrees Celsius, once the cells output falls between 3.5V-3.4V, the available power dissipated along the resistance will be insufficient to maintain a temperature above 175 degrees Celsius. Under these conditions, it would be necessary to stop the usage of the respirator before the full capacity of the cells has been used. The cells would continue to provide power with no negative effect on their lifespan down to an output of 3V.

Introducing a circuit that controls and raises tension as needed would provide a constant tension to the resistance (6) of the thermal treatment reactor (4) over the entire interval of 4.2V-3V of the cells' optimal power output. This ensures that the dissipated power from the resistance remains constant until the full cell capacity is used.

The stable tension provided by the tension control circuit can be dialed in using a potentiometer to a value above the sum of the cell tensions at the beginning of the discharge cycle, so to higher than 8.4V (4.2+4.2). The constant value of the tension control circuit is correlated with the reactor's resistance, which ensures that the necessary power is dissipated to keep the temperature over 175 degrees Celsius over the entire discharge cycle of the cells.

C. The flexible tube (3) with a 32 mm diameter serves to funnel air to the full face mask (11) and to dissipate heat, cooling the sterilized air flowing into the mask by an additional 2-3 degrees Celsius. Thus, the sterilized air reaches the mask at a temperature that is only 2-3 degrees above the ambient temperature. This improves breathing comfort in comparison to filtration masks. The temperature of the sterilized air funneled into the mask is lower than the temperature of the air found between a normal textile surgical mask and a user's face. In a regular filtration mask, the air exhaled at 37 degrees raises the temperature of the air on the next inhale, with the mask's fibers operating as a heat exchanger.

The flexible tube funnels the sterilized air to a full-face mask (11) with a silicone rubber seal and a transparent polycarbonate visor. The internal space of the mask (11) is divided in two zones by a flexible rubber wall that separates the nose and mouth area from the rest of the face. The wall is fitted with 2 one-way valves that only allow air to circulate from the mask's intake near the forehead, down to the mouth and nose area. This separation in two zones is necessary to reduce the space in which expelled air could mix with the new fresh air and cause build-up of CO2 inside the mask. The mouth and nose area has another one-way valve to allow for expelled air to exit the mask, while preventing any ambient infectious air from entering.

FIG. 4 shows the second design version for the portable air sterilizer, in which the air thermal treatment module (1) is mounted directly to the top side of the full face mask (11). In this version, the thermal treatment reactor (4) is surrounded by the spiral heat exchanger (5) and also flanked top and bottom by another plate heat exchanger, also operating in counter-current.

FIG. 5 shows vertical and longitudinal sections through the treatment module (1), in the second design version of this invention. The counter-current channels of the spiral heat exchanger are formed from the aluminum sheets (9) as well as aluminum end caps (12). The plate heat exchanger is formed by the aluminum end caps (12) and two additional aluminum plates (13). Two counter-current air channels are thus formed on each of the spiral heat exchangers' (5) two faces. The air channels adjacent to the spiral heat exchanger (5) are intake channels for the ambient cool infected air. The cool air inhaled through these channels is preheated by the aluminum end caps (12) of the spiral heat exchanger (5) and passes thorough circular orifices (15) to reach the antechamber (16) of the spiral heat exchanger (5). From there, it will follow the other air channel (8) into the thermal reactor (4). Having been treated in the thermal reactor chamber (4), the air continues along the other air channel (8), counter-current to the intake air to which it transfers heat. After this partial cooling, the treated air reaches the sterilized air reception chamber (19) and flows through the cylindrical channels (18) into the main sterilized air channel (17). Having reached the main sterilized air channel (17), the sterilized air is further cooled by giving up heat to the non-treated air flowing counter-current in the intake channels (14). Once cooled, the sterilized air is led through the fixation device (20) into the full-face mask (11).

The respirator with the air sterilization device described in this patent has the following benefits compared to existing respirators on the market:

1) The device provides users with sterilized air after deactivating 99.99% of the viruses. The concentration of viruses remaining active is approximately a hundred times lower than that of viruses able to pass through N95 electret-type filtration masks, which have been considered the most effective.
2) Medical and front-line personnel benefit from complete protection on all three main paths the viruses could enter the body (eyes, nose and mouth).
3) The device does not include disposable components, which could pose environmental safety challenges.
4) The rechargeable power source is light and small in size. The use of the tension regulation and lift circuit guarantees the full use of power cells capacity. Under these operating conditions the cells provide 500-1000 discharge and recharge cycles.
5) The use of thermal stress as a virus inactivation method is very safe and causes no toxic by-products in the sterilized air flow that could be harmful or irritant to the user.
6) This proposed air sterilization design allows for the construction of the first portable full face respirator that uses thermal stress as an aerosolized virus inactivation method. The following factors contributed to portability:

a. The spatial configuration between the thermal treatment reactor (4) and the spiral heat exchanger (5) with the heat reactor being surrounded by the heat exchanger;

b. The very low thermal inertia of the device, resulting from the way the reactor-exchanger system was designed

c. The use of the tension regulation and lift circuit to ensure optimal usage of the power delivered from the rechargeable power cells.

### SUMMARY

We describe a portable air sterilization device that attaches to respirators, and is used for the personal protection of individuals working in highly infectious environments for extended periods of time. The air sterilization device is portable and deactivates 99.99%-100% of aerosolized viruses and bacteria present in the infected air. To sterilize the infected air, the device uses the thermal stress method (Grinshpun et al., 2010).

The device is composed of:
Air treatment module (1)
Electrical power source (2) and
Flexible hose (3) to feed air to the full-face mask (11)

The thermal air treatment module (1) is comprised of an air treatment heat reactor (4) that is included inside a spiral counter-current heat exchanger (5). Through this spatial configuration between the thermal reactor (4) and the spiral heat exchanger (5), the air is cooled after the heat treatment, while the heat is recovered and heat loss into the environment is minimized. The recovered heat is then transferred to the infected air to pre-heat it, prior to it reaching the thermal treatment reactor (4). Thermal sterilization of the air is achieved by heating the air to over 175 degrees Celsius for a duration of 0.3-0.5 seconds.

Other air sterilization models using thermal stress have been proposed (U.S. Pat. Nos. 5,874,050 and 7,332,140B2). Their mass and their necessary electrical inputs do not enable their use as portable devices. Portable devices for air purification by heat treatment are also known: U.S. Pat. Nos. 6,488,900B1 and 9,968,809B2. However, the design choices for the thermal reaction chamber and the heat exchanger unit in the devices presented in the above patents have significant thermal inertia and heat losses, leading to an increased need for a heavy source of electric energy. This caused the above patents to fail in becoming viable commercial products.

1) The air sterilization device, designed to attach to a personal respirator, is composed of: a thermal treatment module (1), an electrical power source (2), and a sterilized air feeding hose (3). The thermal treatment module (1) includes a thermal treatment reactor (4), that is surrounded by a spiral counter-current heat exchanger (5), positioned in a spatial configuration that significantly reduces heat losses and eliminates the need for thermo-isolating mass.

2) The air sterilization device, as in claim 1, is characterized by corrugation in the spiral heat exchanger, such that the corrugation is used on the aluminum walls (9), thus reducing thermal inertia of the device, and achieving a turbulent air flow which further enhances the efficiency of the heat exchange.

3) The air sterilization device, as in claim 2, features a plate counter-current heat exchanger that includes two heat exchangers that surround the thermal treatment reactor (4) on all sides, in such a spatial configuration as to eliminate heat losses and the need for thermo-isolating mass.

4) The air sterilization device, as in claim 3, wherein the electrical power source (2) includes rechargeable power cells, and wherein the electrical power source (2) also uses a circuit to regulate and lift tension as needed to ensure constant tension and energy dissipation along the thermal treatment reactor (4), and a resistance (6) throughout the rechargeable power cells' full discharge cycle.
